(19) United States
(12) Patent Application Publication
(10) Pub. No.: US 2007/0238652 A1
(43) Pub. Date: Oct. 11, 2007

(54) Neutralization of bacterial spores

(75) Inventors: John F. Kokai-Kun, Frederick, MD (US); James J. Mond, Silver Spring, MD (US); Jonathan de la Harpe, New York, NY (US)

Correspondence Address: Medlen & Carroll, LLP, Suite 350, 101 Howard Street, San Francisco, CA, 94105, US

(73) Assignee: Biosynexus Incorporated, Gaithersburg, MD (US)

(21) Appl. No.: 11/704,070

(22) Filed: Feb. 8, 2007

Related U.S. Application Data
(60) Provisional application No. 60/771,322, filed on Feb. 8, 2006.

(51) Int. Cl. A61K 38/00; A61K 31/165; A61K 31/335; A61K 38/16; A62B 27/00; A61P 31/04; A61K 31/43; A61K 31/495

(52) U.S. Cl. 514/8; 128/203.12; 514/12; 514/192; 514/252.12; 514/450; 514/619; 514/786

(57) ABSTRACT

The present invention relates to the field of bacteriology. In particular, the present invention provides compositions (e.g., a lantibiotic-based spore decontaminant (e.g., comprising nisin)) and methods of neutralizing (e.g., killing or inhibiting growth or inhibiting germination of) bacteria (e.g., cells and spores). For example, the present invention provides nisin-based compounds (e.g., for bacterial spore decontamination) and methods of using the same in research, therapeutic and drug screening applications.

This invention claims priority to U.S. Provisional Patent Application No. 60/771,322 filed Feb. 8, 2006, hereby incorporated by reference in its entirety.

### FIELD OF THE INVENTION

The present invention relates to the field of bacteriology. In particular, the present invention provides compositions (e.g., a lantibiotic-based spore decontaminant (e.g., comprising nisin)) and methods of neutralizing (e.g., killing or inhibiting growth or inhibiting germination of) bacteria (e.g., cells and spores). For example, the present invention provides nisin-based compounds (e.g., for bacterial spore decontamination and/or neutralization) and methods of using the same in research, therapeutic and drug screening applications.

### BACKGROUND OF THE INVENTION

An attack or terrorist event using spores of Bacillus anthracis continues to be an imminent threat. In the event of such an attack, many surfaces including human skin will become contaminated with disseminated spores. Spores carried on human skin not only pose the threat of developing into cutaneous anthrax, but can also be carried to other locations distant from the attack site thereby leading to further spore dissemination. This can be particularly problematic if spore contaminated individuals are transported to locations like hospitals where spores shed from the skin may affect debilitated individuals.

A lesson learned from the attacks of 2001 is that the infectious dose of anthrax spores may be much lower than originally believed and spores shed from human skin could spread disease far beyond the initial location of an attack.

Currently, treatments are not available that are designed to decontaminate (e.g., neutralize and/or prevent the growth or germination of) anthrax spores on human skin or other human surfaces (e.g., lungs or hair). Thus, there is a need for compositions and methods that can neutralize and prevent the outgrowth of spores of Bacillus anthracis. Such an agent would ideally be easily disseminated, not be harmful to human surfaces (e.g., skin or lungs) and would be capable of altering (e.g., inhibiting) spore germination and growth potential (e.g., thereby leaving the spores inert and non-infectious).

### DESCRIPTION OF THE DRAWINGS

FIG. 1 shows spore neutralizing activity of nisin on B. anthracis (Ames) spores.

FIG. 2 shows nisin treated spores are attenuated in a mouse pulmonary challenge model.

FIG. 3 shows nisin neutralizes B. anthracis spores dried on a plate. Vegetative growth of buffer treated (a) Sterne and (c) Cipro-R Sterne spores, and (b) nisin pretreated Sterne spores and (d) nisin pretreated Cipro-R Sterne spores.

FIG. 4 shows that nisin penetrates macrophages to neutralize phagocytosed B. anthracis spores. Microscopic images show (a) control cells not treated with nisin and (b) macrophages treated with nisin five hours after phagocytosis of spores.

FIG. 5 shows that nisin can used as a post spore exposure treatment to treat spores in vivo.

FIG. 6 shows the percent survival of mice challenged with control or nisin-treated B. anthracis spores.

FIG. 7 shows Table 1 described in Example 1.

FIG. 8 shows Table 2 described in Example 1.

FIG. 9 shows Table 3 described in Example 2.

FIG. 10 shows Table 4 described in Example 3.

FIG. 11 shows Table 5 described in Example 9.

FIG. 12 shows Table 6 described in Example 4.

### DEFINITIONS

As used herein, the term “subject” refers to an individual (e.g., human, animal, or other organism) to be treated by the methods or compositions of the present invention. Subjects include, but are not limited to, mammals (e.g., murines, simians, equines, bovines, porcines, canines, felines, and the like), and most preferably includes humans. In the context of the invention, the term “subject” generally refers to an individual who will receive or who has received treatment for a condition characterized by the presence of bacteria (e.g., Bacillus anthracis (e.g., in any stage of its growth cycle), or in anticipation of possible exposure to bacteria. As used herein, the terms “subject” and “patient” are used interchangeably, unless otherwise noted.

The term “diagnosed,” as used herein, refers to the recognition of a disease (e.g., caused by the presence of pathogenic bacteria) by its signs and symptoms (e.g., resistance to conventional therapies), or genetic analysis, pathological analysis, histological analysis, and the like.

As used herein the term, “in vitro” refers to an artificial environment and to processes or reactions that occur within an artificial environment. In vitro environments include, but are not limited to, test tubes and cell cultures. The term “in vivo” refers to the natural environment (e.g., an animal or a cell) and to processes or reaction that occur within a natural environment.

As used herein, the terms “attenuate” and “attenuation” used in reference to a feature (e.g. growth) of a bacterial cell or a population of bacterial cells refers to a reduction, inhibition or elimination of that feature, or a reducing of the effect(s) of that feature. For example, when used in reference to a pathogen (e.g., B. anthracis), attenuation generally refers to a reduction in the virulence of the pathogen. Attenuation of a pathogen is not limited to any particular mechanism of reduced virulence. In some embodiments, reduced virulence maybe achieved by neutralization (e.g., inhibiting the growth potential of spores) of the pathogen. In some embodiments, attenuation refers to a feature (e.g., virulence of a population of cells or spores). For example, in some embodiments of the present invention, a population of pathogen cells or spores is treated (e.g., using methods and compositions of the present invention) such that the population is decreased in virulence.

As used herein, the term “virulence” refers to the degree of pathogenicity of a microorganism (e.g., as indicated by the severity of signs and symptoms of the disease produced or its ability to invade the tissues of a subject). It is generally measured experimentally by the median lethal dose (LD₅₀) or median infective dose (ID₅₀). The term may also be used to refer to the competence of any infectious agent to produce pathologic effects.

As used herein, the terms “neutralize” and “neutralization” when used in reference to bacterial cells or spores (e.g. B. anthracis cells and spores) refers to an inhibition of the ability of the spores to germinate and/or cells to grow (e.g., although an understanding of the mechanism is not necessary to practice the present invention and the present invention is not limited to any particular mechanism of action, in some embodiments, neutralization results from a termination of germination of spores, whereas, in other embodiments, neutralization results from killing of the cells and/or spores). In preferred embodiments of the present invention, compositions comprising nisin are used to neutralize (e.g., inhibit the germination and outgrowth potential of) bacterial cells or spores (e.g., B. anthracis cells and spores).

As used herein, the term “lantibiotic-based spore decontaminant” refers to a composition comprising a lantibiotic that is configured to neutralize bacterial spores (e.g., B. anthracis spores) that are present on and/or in a subject (e.g., a human subject). Thus, a lantibiotic-based spore decontaminant is an agent that is configured specifically for administration (e.g., via topical, mucosal or internal routes) to a subject (e.g., human subject), preferably without the decontaminant harming (e.g., being irritating or damaging to) the subject. The present invention is not limited by the lantibiotic used. In some preferred embodiments, the lantibiotic is nisin or from the nisin family of lantibiotics. Various examples and formulations of a lantibiotic-based spore decontaminant are provided herein.

As used herein, the term “effective amount” refers to the amount of a composition (e.g., a lantibiotic-based spore decontaminant) sufficient to effect a beneficial or desired result (e.g., bacterial cell killing or neutralization (e.g., neutralization of B. anthracis spores)). An effective amount can be administered in one or more administrations, applications or dosages and is not intended to be limited to a particular formulation or administration route.

As used herein, the term “administration” refers to the act of giving a drug, prodrug, or other agent, or therapeutic treatment (e.g., a composition of the present invention) to a physiological system (e.g., a subject or in vivo, in vitro, or ex vivo cells, tissues, and organs). Exemplary routes of administration to the human body can be through the eyes (ophthalmic), mouth (oral), skin (transdermal, or topical), nose (nasal), lungs (inhalant), mucosal (e.g., oral mucosa or buccal), rectal, ear, by injection (e.g., intravenously, subcutaneously, intratumorally, intraperitoneally, etc.) and the like.

As used herein, the term “treating a surface” refers to the act of exposing a surface to one or more compositions of the present invention. Methods of treating a surface include, but are not limited to, spraying, misting, submerging, wiping, and coating. Surfaces include organic surfaces (e.g., food products, surfaces of animals, etc.) and inorganic surfaces (e.g., medical devices, countertops, instruments, articles of commerce, clothing, etc.).

As used herein, the term “co-administration” refers to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s).

As used herein, the terms “contact” or “contacting” refer to any manner in which a composition of the present invention (e.g., a solution or cream comprising a lantibiotic-based spore decontaminant of the present invention) is brought into a position where it can mediate or alter (e.g., inhibit) germination and/or growth of a bacterial cell and/or spore. For example, “contacting” may comprise any of the methods of administration or methods of treating a surface mentioned herein.

As used herein, the terms “signs and symptoms of B. anthracis infection” and “signs and symptoms of anthrax” refer to any one of a number of characteristics displayed by a subject (e.g., a human subject or other mammal) that has been infected with B. anthracis. Signs and symptoms may include, for example, cold or flu-like symptoms (e.g., for several days), respiratory problems (e.g., mild to severe), cutaneous symptoms like eschar formation, and other characteristics recognized by medical persons (e.g., doctors, nurses, etc.) as those displayed by a subject with B. anthracis infection.

As used herein, the term “pharmaceutical composition” refers to the combination of an active agent (e.g., a composition comprising a lantibiotic-based spore decontaminant and/or neutralizer) with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions (e.g., toxic, allergic, or immunological reactions) when administered to a subject.

As used herein, the term “topically” refers to application of the compositions of the present invention to the surface of the skin or mucosal cells and tissues (e.g., alveolar, buccal, lingual, masticatory, or nasal mucosa, and other tissues and cells that line hollow organs or body cavities).

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintigrants (e.g., potato starch or sodium starch glycolate), and the like. The compositions also may include stabilizers and preservatives. Examples of carriers, stabilizers, and adjuvants are described in the art (See e.g., Martin, Remington's Pharmaceutical Sciences, 15th Ed., Mack Publ. Co., Easton, Pa. (1975), incorporated herein by reference).

As used herein, the term “pharmaceutically acceptable salt” refers to any salt (e.g., obtained by reaction with an acid or a base) of a compound of the present invention that is physiologically tolerated in the target subject (e.g., a mammalian subject, and/or in vivo or ex vivo, cells, tissues, or organs). “Salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, the term “therapeutically effective amount” (e.g., of a composition comprising a lantibiotic-based spore decontaminant or neutralizer) refers to the amount (e.g., of a composition comprising nisin) that is effective to treat or prevent pathological conditions (e.g., signs and symptoms of disease) associated with B. anthracis infection (e.g., germination, growth, toxin production, etc.) in a subject.

As used herein, the term “medical devices” includes any material or device that is used on, in, or through a subject's or patient's body, for example, in the course of medical treatment (e.g., for a disease or injury). Medical devices include, but are not limited to, such items as medical implants, wound care devices, drug delivery devices, and body cavity and personal protection devices. Medical implants include, but are not limited to, urinary catheters, intravascular catheters, dialysis shunts, wound drain tubes, skin sutures, vascular grafts, implantable meshes, intraocular devices, heart valves, and the like. Wound care devices include, but are not limited to, general wound dressings, biologic graft materials, tape closures and dressings, and surgical incise drapes. Drug delivery devices include, but are not limited to, needles, drug delivery skin patches, drug delivery mucosal patches and medical sponges. Body cavity and personal protection devices, include, but are not limited to, tampons, sponges, surgical and examination gloves, and toothbrushes.

As used herein, the term “therapeutic agent” refers to a composition that decreases the infectivity, morbidity, or onset of mortality in a subject contacted by a pathogenic microorganism or that prevent infectivity, morbidity, or onset of mortality in a host contacted by a pathogenic microorganism. Therapeutic agents encompass agents used prophylactically (e.g., in the absence of a pathogen) in view of possible future exposure to a pathogen. Such agents may additionally comprise pharmaceutically acceptable compounds (e.g., adjuvants, excipients, stabilizers, diluents, cofactors and the like). In some embodiments, the therapeutic agents of the present invention are administered in the form of topical compositions, injectable compositions, ingestible compositions, inhalable compounds and the like. When the route is topical, the form may be, for example, a solution, cream, ointment, salve or spray.

As used herein, the term “cofactor” is a compound that enhances the desired activity of a composition (e.g., nisin) such that a desirable outcome is increased by the addition of the cofactor. “Cofactors” include but are not limited to, detergents (e.g., Tween-20, or carvacol), chaotropic agents, essential oils (e.g., tea tree oil), chelators (e.g., EDTA), solubility enhancers (e.g., chitosan), and absorption enhancers. Addition of such cofactors to a composition increase the over efficacy of the composition.

As used herein, the term “pathogen” refers a biological agent that causes a disease state (e.g., infection, sepsis, etc.) in a host. “Pathogens” include, but are not limited to, viruses, bacteria (e.g., Bacillus anthracis), archaea, fungi, protozoans, mycoplasma, prions, and parasitic organisms.

The terms “bacteria” and “bacterium” refer to all prokaryotic organisms, including those within all of the phyla in the Kingdom Procaryotae. It is intended that the term encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc. In some embodiments, bacteria are continuously cultured. In some embodiments, bacteria are uncultured and existing in their natural environment (e.g., at the site of a wound or infection) or obtained from patient tissues (e.g., via a biopsy). Bacteria may exhibit pathological growth or proliferation. As used herein, the term “microorganism” refers to any species or type of microorganism, including but not limited to, bacteria, archaea, fungi, protozoans, mycoplasma, and parasitic organisms.

As used herein, the term “non-human animals” refers to all non-human animals including, but not limited to, vertebrates such as rodents, non-human primates, ovines, bovines, ruminants, lagomorphs, porcines, caprines, equines, canines, felines, aves, etc.

As used herein, the term “kit” refers to any delivery system for delivering materials. In the context of therapeutic agents (e.g., compositions comprising nisin), such delivery systems include systems that allow for the storage, transport, or delivery of therapeutic agents and/or supporting materials (e.g., written instructions for using the materials, etc.) from one location to another. For example, kits include one or more enclosures (e.g., boxes) containing the relevant therapeutic agents and/or supporting materials. As used herein, the term “fragmented kit” refers to delivery systems comprising two or more separate containers that each contain a subportion of the total kit components. The containers may be delivered to the intended recipient together or separately. For example, a first container may contain a composition comprising nisin for a particular use, while a second container contains a second agent (e.g., an antibiotic or spray applicator). Indeed, any delivery system comprising two or more separate containers that each contains a subportion of the total kit components are included in the term “fragmented kit.” In contrast, a “combined kit” refers to a delivery system containing all of the components of a therapeutic agent needed for a particular use in a single container (e.g., in a single box housing each of the desired components). The term “kit” includes both fragmented and combined kits.

### DETAILED DESCRIPTION OF THE INVENTION

Anthrax remains a top bioterrorism/biowarfare threat. Spores of Bacillus anthracis are relatively easy to produce and disseminate and the “weaponization” process makes these spores even more effective bioterrorism agents. One of the lessons learned from the events of 2001 is that the infectious dose of anthrax spores, especially weaponized spores, may be much lower than originally believed. The infectious dose for spores was thought to be ˜10,000 (See, e.g., Frist et al., Rowman & Littlefield Publishers (2002)) which would allow spores to infiltrate deeply into the lungs and penetrate the alveoli where the spores are taken up by alveolar macrophages (See, e.g., Dixon et al., Science 2:453-463 (2000)). The deaths of two victims from pulmonary anthrax in 2001 in the absence of detectable spores in their surroundings suggests that the infectious dose of Bacillus anthracis is lower than that previously suspected. For example, one explanation for these two deaths is that these two victims came into contact with minute quantities of spores that were contaminants on their mail and these spores efficiently reached deep into the alveoli.

In the case of exposure to anthrax spores through aerosolization, spores can be recovered from many surfaces including human skin, and affected persons could act as carriers of spores to other sites beyond the initial area of attack, leading to secondarily infected persons. This effect could be particularly devastating if the original victim(s) are transported to a setting where there are debilitated persons such as a hospital.

Spores on skin may also cause cutaneous anthrax (See, e.g., Mock et al., Annu. Rev. Microbiol. 55:647-671 (2001)), which if untreated can result in severe local edema associated with fever and malaise and occasionally systemic anthrax. Skin contamination also increases the possibility of self-infecting via the oral route which leads to gastrointestinal or oropharyngeal anthrax (See, e.g., Mock et al., Annu. Rev. Microbiol. 55:647-671 (2001)). Thus, in the event of an attack with anthrax spores, there is the immediate threat to the people involved in the initial exposure and there also remains the secondary threat of further dissemination of spores carried on the skin of the affected people to other locations. Simply employing a decontamination shower at the site of attack only serves to further spread of infective spores (e.g., in the water run-off) and does not neutralize spores. Transporting people who carry spores on their skin to a hospital could introduce infectious spores into an environment where potentially hundreds of people could be infected by secondary exposure. Thus, prior to any transport of victims of an anthrax spore attack, it would be critical to neutralize any spores that could be carried (e.g., on the skin, hair or other non-human surfaces).

Currently, there are no products specifically designed to neutralize spores on human skin (See, e.g., Sagripanti et al., Appl. Environ. Microbiol. 65:4255-4260 (1999)). Neutralization procedures recommend using at least a 10% bleach solution to decontaminate surfaces, but the Environmental Protection Agency does not recommend the use of bleach on skin for toxicity reasons. Furthermore, bleach poses serious toxicity hazards to the skin of infants and to the mucous membranes and eyes of adults. In a recent study of hand hygiene using commercially available hand antiseptics, no intervention was found to reduce spore contamination by more than 2 log₁₀ (See, e.g., Weber et al., JAMA, 289:1274-1277 (2003)). Additionally, waterless rubs were found to be ineffective against spores.

One study has reported on the ability of several commercially available products to neutralize spores of B. subtilis (See, e.g., Sagripanti et al., AOAC Int. 80:1198-1207 (1997)). In this study, preparations of gluteraldehyde, formaldehyde, hydrogen peroxide, peracetic acid, cupric ascorbate with hydrogen peroixide, sodium hypochlorite, and phenols were analyzed, with only hypochlorite, peracetic acid and cupric ascorbate achieving >99% inactivation of spores of B. subtilis after 30 minutes exposure, while exposure to the other preparations resulted in only 10%-0% of spore neutralization. Similar types of studies have shown that there is a 90-100% loss of sporicidal activity for these reagents (except for gluteraldehyde) in the presence of serum levels as low as 1%. (See, e.g., Sagripanti et al., AOAC Int. 80:1198-1207 (1997)). Furthermore, commercial and household products were used following the manufacturer's instructions, with LYSOL and CLOROX showing reductions in spore counts of 90% and 99% respectively in the absence of organic contaminants (See, e.g., Sagripanti et al., Appl. Environ. Microbiol. 65:4255-4260 (1999)). One of the most effective products tested was RENALIN (20% H₂O₂ and 4% peracetic acid) which achieved a 4-5log₁₀ reduction in spore count, but required an 11 hour exposure time. Furthermore, peracetic acid is explosive and is irritating to the skin.

Decontaminants that are harsh on the skin may have the added detriment of causing breaks in the skin possibly leading to the development of cutaneous anthrax if all of the spores are not neutralized. Some of the more recent approaches to directly treat spores such as the lytic phage enzymes and high energy microparticle emulsions also have inherent limitations such as the need to pre-germinate the spores in order for these treatments to be effective.

Thus, in order to address the dissemination of spores (e.g., of B. anthracis spores intentionally used in a terrorist attack), compositions and methods will need to be orders of magnitude better than those previously mentioned. Thus, an immediate need exists for agents that are specifically designed to neutralize anthrax spores (e.g., on human surfaces such as skin, hair, wounds, etc.). Such an agent should be gentle to skin and amenable to being formulated in various ways (e.g., wipes, sprays, foams, etc. that can be used in the field in situations where anthrax spore exposure is suspected as an additional layer of protection (e.g., beyond the use of antibiotics and post exposure vaccinations)).

After spores are inhaled, they progress to the bronchial alveola and are phagocytosed by alveolar macrophages. It is within these macrophages that the spores begin to germinate and grow as vegetative cells. It would be an advantage in treatment to be able to neutralize inhaled spores either prior to phagacytosis by macrophages, or while the macrophages are still present in the lungs and prior to spore germination. The present invention provides such an opportunity to disrupt the pathogenic progression of spores to vegetative cells through the demonstrated capacity to neutralized spores post inhalation and post phagocytosis.

Accordingly, the present invention provides lantibiotic- (e.g., nisin-) based compositions and methods of using the same for neutralizing (e.g., killing or inhibiting growth or inhibiting germination of) bacterial cells and spores (e.g., B. anthracis cells and spores). For example, the present invention provides therapeutic agents (e.g., a lantibiotic-based spore decontaminant or neutralizer) and methods of using the same in research, preventative, therapeutic and drug screening applications.

Nisin is an antimicrobial substance produced by Lactococcus lactis. It is a member of a group of similar substances referred to as lantibiotics, which include subtilin, epidermin, gallidermin, pep 5, cinnamycin, lacticin 481, duramycin and ancovenin. Nisin is a peptide comprised of 34-amino acid residues and contains five ring structures cross-linked by thioether bridges that form lanthionine or β-methyllanthionine. Formulations of nisin are described in U.S. Pat. Nos. 5,135,0910 and 5,753,614, herein incorporated by reference in their entireties. Variants of nisin are described in U.S. Pat. No. 6,448,034, herein incorporated by reference in its entirety. Additional lantibiotics similar to nisin are described in U.S. Pat. Nos. 5,594,103 and 5,928,146, herein incorporated by reference in their entireties. Lantibiotics can be further subdivided into families (e.g., nisin is in the Type-A(I) lantibiotic family which also includes subtilin, epidermin, gallidermin, mutacin, pep5 epicidin and epilancin).

Nisin has broad-spectrum activity against gram positive bacteria and some activity against gram negative bacteria. Blackburn et al. (U.S. Pat. No. 5,866,539, the contents of which are incorporated in their entirety by reference) generally describes use of nisin along with anti-bacterial agents to treat skin infections. Furthermore, U.S. Pat. App. No. 20040192581, hereby incorporated by reference in its entirety for all purposes, also describes topical administration of nisin.

Nisin has been used as a food preservative (See, e.g., Hansen et al., Crit. Rev. Food Sci., Nutr. 31:69-93 (1994)) and has received a “Generally Recognized As Safe” (GRAS) designation by the Food and Drug Administration (See, e.g., Food and Drug Administration, Code of Federal Regulations 21:524 2001; Food and Drug Administration, Fed. Regist. 53:11247-11251 1998). Commercial use by the food processing industry employs a nisin preparation comprising ˜2.5% nisin.

One target for nisin is the cytoplasmic membrane of bacteria where it acts to dissipate the proton motive force through formation of pores in the cytoplasmic membranes (See, e.g., McAuliffe et al., FEMS Microbiol. Rev. 25:285-308 (2001)). Nisin is believed to form pores in vegetative bacteria in two different ways. In an artificial membrane, sufficient concentrations of nisin form homogenous nisin pores, but in the bacterial cytoplasmic membrane, nisin interacts with lipid II (e.g., undecaprenyl-pyrophosphoryl-MurNAc-(pentapeptide)-GlcNAc) to form heterologous pores (See, e.g., McAuliffe et al., FEMS Microbiol. Rev. 25:285-308 (2001); Wiedemann et al., Biolog. Chem. 276:1772-1779 (2001)). At lower concentrations, nisin also inhibitis cell wall biosynthesis by binding to lipid II and inhibiting its incorporation into the peptidoglycan network (See, e.g.; Wiedemann et al., Biolog. Chem. 276:1772-1779 (2001)). Nisin interacts with spores of Clostridial and Bacillus species. Interaction between nisin and the spores appears to involve the reactive double bond in the DHA residue at position 5 and sulfhydral groups on spores (See, e.g., Chan et al., Appl. Environ. Microbiol. 62:2966-2969 (1996); Delves-Broughton et al., Antonie van Leeuwenhoek 69:202 (1996); Morris et al., Biolog. Chem. 259:13590-13594 (1984); Pol et al., Appl. Environ. Microbiol. 67:1693-1699 (2001)). Other lantibiotics (e.g., subtilin) also have anti-spore activity. This interaction results in a termination of the germination process in which the spore can be observed changing from phase bright to phase dark as it takes on water (See, e.g., Setlow et al., Curr. Opinion Microbiol. 6:550-556 (2003)), when viewed under phase microscopy and then stopping, remaining suspended in phase dark indefinitely. However, it has heretofore remained unknown and untested as to whether lantibiotics (e.g., nisin) can treat existing exposure to bacterial spores (e.g., whether a lantibiotic could successfully treat (e.g., inhibit germination or growth of) B. anthracis spores in vivo or on the skin or on mucosal surfaces).

The initial interaction between B. anthracis spores and a human host occurs when spores are engulfed by regional macrophages at the point of entry (e.g., in the lungs by alveolar macrophages in the case of inhalation exposure) (See, e.g., Dixon et al., Science 2:453-463 (2000)). The phagocytosed spores begin to germinate within the macrophages en route to the regional lymph nodes (See, e.g., Guidi-Rontani et al., Trends Microbiol. 10:405-409 (2002)). This germination occurs when signal, including small molecules, bind to membrane-associated protein receptors and induce the dormant spores to return to vegetative growth (See, e.g., Ireland et al., Bacteriol. 184:1296-1303 (2002)). As the bacteria begin to grow as vegetative cells, they escape the macrophage to become systemic where they can approach levels of ˜10⁸ per ml of blood (See, e.g., Mock et al., Annu. Rev. Microbiol. 55:647-671 (2001)). Anthrax toxins are expressed at high levels during vegetative growth of the bacteria (See, e.g., Abrami et al., Trends Microbiol. 13:72-78 (2005)). Thus, in preferred embodiments, therapeutic or pharmaceutical agents of the present invention (e.g., a lantibiotic-based spore decontaminant for human use) blocks the germination process (e.g., arrests the pathogenesis of B. anthracis at the earliest point in the cycle before spore germination, vegetative cell outgrowth and expression of any toxins). This could occur on the skin, in wounds or in the lungs. In other preferred embodiments, a lantibiotic-based spore decontaminant for human use prevents signs and symptoms of disease caused by B. anthracis. In some embodiments, a therapeutic or pharmaceutical agent (e.g., a lantibiotic-based spore decontaminant for human use) kills vegetative forms of B. anthracis (See Examples 3, 5 and 6).

The safety profile of nisin has been extensively studied including topical safety due to its use in three commercial topical veterinary products (See, e.g., Sears et al., Dairy Sci. 75:3185-3190 (1992)). These products are used in the dairy industry to sanitize cows' teats and to prevent spoilage organisms from entering the milk supply. The use of nisin-based topical treatments also prevents the infection of the bovine mammary glands (mastitis). Nisin for use in the dairy industry has been formulated as a rapidly-acting teat dip solution, a barrier gel designed to provide protection from pathogen infections in severe conditions of moisture and cold, and an antimicrobial moist paper wipe. Nisin-based topical sanitizing products have been marketed to the dairy industry for almost 15 years and have been shown to be non-irritating to both cow's teats and the operators hand, and to be effective against a variety of other pathogens (e.g., E. coli, S. aureus, S. epidermidis, K. pneumoniae, S. agalactiae and S. uberis (See, e.g., Sears et al., Dairy Sci. 75:3185-3190 (1992)). The GRAS status of nisin is also relevant in regards to the topical use of nisin as people have been known to ingest semi-solid dosage forms of drugs, and if used on the face, accidental ingestion of a topical application of nisin would not be a concern. The safety of nisin for intravenous use has also been shown to be safe at moderate doses (See, e.g., Goldstein et al., Antimicro. Chemother. 42:277-278 (1998)).

In preferred embodiments, resistance to a nisin-based spore decontaminant for human use does not develop. Although an understanding of the mechanism is not necessary to practice the present invention and the present invention is not limited to any particular mechanism of action, in some embodiments, because nisin need not be administered to replicating cells (e.g., nisin is effective at neutralizing non-replicating cells or spores (e.g., those that are not undergoing active metabolism)), there is no active mechanism of selection of nisin resistance for spores treated with nisin. Furthermore, because anthrax, as a disease, is not passed from person to person, random mutation of a spore resistant to nisin is highly unlikely to be selected by nisin treatment (e.g., any nisin resistant spore variant that might occur will remain isolated in the primary host and not be spread to secondary hosts).

The present invention is not limited by the particular formulation of a therapeutic agent (e.g., lantibiotic- (e.g., nisin-) based spore decontaminant or neutralizer (e.g., for human use)) of the present invention. Indeed, a therapeutic agent (e.g., a lantibiotic-based spore decontaminant for human use) of the present invention may comprise one or more different agents in addition to the lantibiotic (e.g., nisin). These agents or cofactors include, but are not limited to, surfactants, additives, buffers, solubilizers, chelators, oils, salts, antibacterials, and other agents including combinations of other lantibiotics or antimicrobial peptides. In preferred embodiments, a therapeutic agent (e.g., a lantibiotic-based spore decontaminant (e.g., for human use)) of the present invention comprises a combination of agents and/or co-factors that enhance the lantibiotic's (e.g., nisin's) spore neutralizing activity. In some preferred embodiments, the presence of one or more co-factors or agents reduces the amount (e.g., reduces the MIC) required for effective spore (B. anthracis spore) neutralization. In some preferred embodiments, the chelator comprises EDTA. In some embodiments, the surfactant comprises a detergent polysorbate (e.g., PEG(20)sorbitan monolaurate, polyoxyethylenesorbitan monolaurate, Tween-20, Tween-80, or other Tween reagent), or essential oils like carvacol. In some embodiments, the buffer is a sodium citrate buffer or contains ZnCl. However, the present invention is not limited by the type of co-factor or agent used in a therapeutic agent of the present invention.

Methods of formulating pharmaceutical compositions are well-known to those of ordinary skill in the art (see, e.g., Remington's Pharmaceutical Sciences, 18.sup.th Edition, Gennaro, ed. (Mack Publishing Company: 1990)). In some embodiments, a lantibiotic-based spore decontaminant of the present invention (e.g., for human use) may comprise pharmaceutically acceptable diluents, preservatives, solubilizers, emulsifiers, adjuvants and/or carriers. Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol), solubility enhancing agents (e.g., chitosan), and bulking substances (e.g., lactose, mannitol); incorporation of the material into particulate preparations of polymeric compounds such as polylactic acid, polyglycolic acid, etc. or into liposomes. Hylauronic acid may also be used (See, e.g., Remington's Pharmaceutical Sciences, 18th Ed. (1990, Mack Publishing Co., Easton, Pa. 18042) pages 1435-1712 which are herein incorporated by reference).

In some embodiments, a lantibiotic-based spore decontaminant of the present invention comprises Tween-20. In some embodiments, Tween-20 is used at a final concentration of between 0.05%-1.0%. In some embodiments, Tween-20 is used at a concentration greater than 1%. In some embodiments, Tween-20 is used at a concentration less than 0.05%. Similarly, in some embodiments, a spore neutralizing therapeutic of the present invention comprises carvacol at a final concentration of between 0.5-5.0%. In some embodiments, carvacol is used at less than 0.5%. In some embodiments, carvacol is used at greater than 5.0%. In some embodiments, a spore neutralizing therapeutic of the present invention comprises chitosan. In some, chitosan is present at a concentration of 0.01 mg/ml or more (e.g., 0.1 mg/ml, 0.2 mg/ml, 0.5 mg/ml or more).

In other preferred embodiments, the present invention provides a lantibiotic-based spore decontaminant comprising nisin. In some embodiments, the decontaminant comprises one or more lantibiotics in addition to nisin (e.g., subtilin). The present invention is not limited by the type of lantibiotic used. Indeed, a variety of lantibiotics are contemplated to be useful in the present invention including, but not limited to, epidermin, gallidermin, gallidermin, mutacin-1140, mut.-III, mut.-B-Ny266, mutacin I, ericin-A, ericin-S, subtilin, nisin, actagardine, mersacidin, plantaricin-c, salivaricin-a, variacin, lacticin-481, mutacin-II, SA-FF22, ltcA1, pln-w-α, ancovenin, duramycin-C, cinnamycin, duramycin, duramycin-B, sublancin, pep-5, epicidin-280, epilancin-k7, lcnA1, pln-W-β, lactocin-S, sapB, and cypemycin (See, e.g., Rink et al., 2005 Biochemistry 44, 8873-8882). In some embodiments, the lantibiotic-based spore decontaminant comprises a modified form of lantibiotic (e.g., a modified nisin (e.g., PEGylated nisin (e.g., nisin comprising a linear or branched form of polyethylene glycol))). In some embodiments, a lantibiotic-based spore decontaminant is administered to a subject under conditions such that bacterial spores are neutralized (e.g., killed, prevented from germinating, or inhibited from vegetative cellular outgrowth). The present invention is not limited by the type of bacterial spore neutralized. In some preferred embodiments, the spore is a Bacillus spore. In further preferred embodiments, the Bacillus spore is a Bacillus anthracis spore. The Bacillus anthracis spore may be a naturally occurring spore or a genetically or mechanically engineered form (e.g., a “weaponized” spore). The spore may also be from an antibiotic resistant strain of B. anthracis (e.g., ciprofloxacin resistant). In some embodiments, a lantibiotic-based spore decontaminant is administered to a subject under conditions such that spore germination or growth is prohibited and/or attenuated. In some embodiments, greater than 90% (e.g., greater than 95%, 98%, 99%, all detectable) of bacterial spores are neutralized (e.g., killed). In some embodiments, there is greater than 2 log (e.g., greater than 3 log, 4 log, 5 log, . . . ) reduction in bacterial spore outgrowth. In some embodiments, the reduction is observed in two days or less following initial treatment (e.g., 20 hours, 18 hours, . . . ). In some embodiments, the reduction is observed in three days or less, four days or less, or five days or less. In some preferred embodiments, reduction in spore outgrowth occurs within hours (e.g., with 1 hour (e.g., in 20-40 minutes or less), within 2 hours, within 3 hours, within 6 hours or within 12 hours). In some preferred embodiments of the invention, spores neutralization (e.g., the inability of the spore to germinate) lasts for at least 3 days, at least 7 days, at least 14 days, at least 21 days, at least 28 days, or at least 56 days.

The present invention demonstrates that a lantibiotic-based spore decontaminant comprising nisin inhibits spore germination in macrophages of a subject (See, e.g., Example 5). Furthermore, the present invention demonstrates that a lantibiotic-based spore decontaminant comprising nisin protects subjects from B. anthracis infection, signs and symptoms of anthrax, and death caused thereby (See, e.g., Example 6). Furthermore, the present invention demonstrates that a lantibiotic-based spore decontaminant comprising nisin effectively neutralizes antibiotic resistant forms of B. anthracis (See, e.g., Example 8). Thus, in some embodiments, the present invention provides a method of protecting a subject exposed to B. anthracis spores from infection (e.g., from displaying signs and symptoms of disease (e.g., anthrax) caused by B. anthracis) comprising administering to said subject a lantibiotic-based spore decontaminant comprising nisin under conditions such that B. anthracis spores are neutralized (e.g., prevented from germinating and growing as vegetative cells).

A lantibiotic-based spore decontaminant (e.g., comprising nisin) of the present invention can be administered to a subject (e.g., to the skin or other surface of a subject (e.g., hair, mucosal surface, airway, or a wound) as a therapeutic or as a prophylactic to prevent bacterial spore germination or growth. It is contemplated that a lantibiotic-based spore decontaminant can be administered to a subject via a number of delivery routes.

For example, the compositions of the present invention can be administered to a subject (e.g., to skin, hair, airway, or to a skin burn or wound surface) by multiple methods, including, but not limited to: being suspended in a solution (e.g., colloidal solution) and applied to a surface; being suspended in a solution and sprayed onto a surface using a spray applicator; being mixed with fibrin glue and applied (e.g., sprayed) onto a surface (e.g., skin); being impregnated onto a wound dressing or bandage and applying the bandage to a surface (e.g., an infection or wound); being applied by a wipe soaked with a therapeutic agent (e.g., a lantibiotic-based spore decontaminant) of the present invention; being applied by a controlled-release mechanism; being impregnated on one or both sides of an acellular biological matrix that can then be placed on a surface (e.g., skin) thereby protecting at both the wound and graft interfaces; being applied as a liposome; or being applied on a polymer.

In some embodiments, a lantibiotic-based spore decontaminant is administered to a subject via submerging the subject's body in a solution comprising a lantibiotic-based spore decontaminant (e.g., in a tub). In some embodiments, a lantibiotic-based spore decontaminant is administered via a shower (e.g., a shower of solution comprising the lantibiotic-based spore decontaminant). For example, in some embodiments, a lantibiotic-based spore decontaminant of the present invention is formulated such that it can be administered to large numbers of people (e.g., 10, 100, 500, 1000, 5000, 10,000 or more) at a single site. In some embodiments, lantibiotic-based spore decontaminants of the present invention are formulated in a concentrated, (e.g., concentrated solid or liquid form (e.g., for transportation ease)) that can be solublized or diluted at any given site (e.g., the site of exposure to B. anthracis spores (e.g., a terrorist attack site)). In some embodiments, a lantibiotic-based spore decontaminant of the present invention is used with a decontamination unit, for example, those described in U.S. Pat. Nos. 4,989,279; 5,544,369; 4,883,512; 5,061,235; 4,858,256; 5,607,652; 4,687,686; and U.S. Pat. App. No. 20040238007, each of which is hereby incorporated by reference.

In some embodiments, subjects that are administered a lantibiotic-based spore decontaminant all receive the decontaminant at the same site (e.g., using a mobile decontamination unit (e.g., a transportable shower configured to dispense (e.g., spray) a decontaminant of the present invention)). In some embodiments, the decontaminant is administered at the site of exposure (e.g., at the site of a terrorist attack or accident). In some embodiments, the decontaminant is administered at a hospital (e.g., in a location designated for decontamination of biological agents). Lantibiotic-based spore decontaminants of the present invention also find use in a research setting. For example, In some embodiments, the decontaminant is used in a research laboratory (e.g., to decontaminate human or animal surfaces (e.g., skin, hair, etc.).

While an understanding of the mechanism is not necessary to practice the present invention and while the present invention is not limited to any particular mechanism of action, it is contemplated that, in some embodiments, once administered to a site (e.g., skin, hair, etc) comprising bacterial spores (e.g., spores of B. anthracis), a lantibiotic-based spore decontaminant of the present invention comes into contact with the spores thereby neutralizing them.

In other embodiments, the compositions and methods of the present invention find application in the treatment of surfaces for neutralizing spores (e.g., B. anthracis spores) thereon. It is contemplated that the methods and compositions of the present invention may be used to treat numerous surfaces, objects, materials and the like (e.g., medical or first aid equipment, nursery and kitchen equipment and surfaces) that have been exposed to bacterial (e.g., B. anthracis) spores in order to neutralize the spores and to control and/or prevent the spread of bacterial exposure.

In other embodiments, the compositions may be impregnated into absorptive materials, such as sutures, bandages, and gauze, a wipe, or coated onto the surface of solid phase materials, such as surgical staples, zippers and catheters to deliver the compositions to a site that may have bacterial (e.g., B. anthracis) spores (e.g., for neutralizing the spores). In some embodiments, a lantibiotic-based spore decontaminant of the present invention is formulated as a moist paper wipe or as a gel (e.g., a barrier gel). Other delivery systems of this type will be readily apparent to those skilled in the art.

Subjects that may be exposed to bacterial (e.g., B. anthracis) spores, and therefore candidates for treatment with compositions and methods of the present invention, are preferably humans. In some embodiments, human subjects are of any age (e.g., adults, children, infants, etc.) that have been exposed to bacterial (e.g., B. anthracis) spores. In some embodiments, the human subjects are subjects that receive a direct exposure to bacterial (e.g., B. anthracis) spores (e.g., via touching the source of the spores (e.g., a contaminated piece of mail) or by inhaling the spores (e.g., spores intentionally released into the air). In some embodiments, the human subjects are subjects that receive exposure to bacterial (e.g., B. anthracis) spores from a source other than the primary source (e.g., via contact with one or more primarily exposed subjects (e.g., emergency persons arriving at a scene of a terrorist attack). Furthermore, subjects may benefit from treatment with a composition of the present invention to any portion of the subject's body. For example, a spray may be used to treat (e.g., coat) any exposed surface (e.g., skin) of the subject. Alternatively, a subject may treat their entire body (e.g., coat their entire body with a lantibiotic-based spore decontaminant of the present invention (e.g., using a shower or tub described herein)). The present invention is not limited to human subjects. Indeed, any animal subject (e.g., dog, cat, horse, etc.) exposed to bacterial (e.g., B. anthracis) spores may benefit from treatment with the compositions of the present invention.

The lantibiotic-based spore decontaminants of the invention may be formulated for administration by any route, such as oral, topical, inhaled or parenteral. The compositions may be in the form of tablets, capsules, powders, granules, lozenges, foams, creams or liquid preparations.

The topical formulations of the present invention may be presented as, for instance, ointments, creams or lotions, foams, eye ointments and eye or ear drops, impregnated dressings and aerosols, and may contain appropriate conventional additives such as preservatives, solvents (e.g., to assist drug penetration), and emollients in ointments and creams.

The topical formulations may also include agents that enhance penetration of the active ingredients through the skin. Exemplary agents include a binary combination of N-(hydroxyethyl)pyrrolidone and a cell-envelope disordering compound, a sugar ester in combination with a sulfoxide or phosphine oxide, and sucrose monooleate, decyl methyl sulfoxide, and alcohol.

Other exemplary materials that increase skin penetration include surfactants or wetting agents including, but not limited to, polyoxyethylene sorbitan mono-oleoate (Polysorbate 80); sorbitan mono-oleate (Span 80); p-isooctyl polyoxyethylene-phenol polymer (Triton WR-1330); polyoxyethylene sorbitan tri-oleate (Tween 85); dioctyl sodium sulfosuccinate; and sodium sarcosinate (Sarcosyl NL-97); and other pharmaceutically acceptable surfactants.

In certain embodiments of the invention, the formulations may further comprise one or more alcohols, zinc-containing compounds, emollients, humectants, thickening and/or gelling agents, neutralizing agents, and surfactants. Water used in the formulations is preferably deionized water having a neutral pH. Additional additives in the topical formulations include, but are not limited to, silicone fluids, dyes, fragrances, pH adjusters, and vitamins.

The topical formulations may also contain compatible conventional carriers, such as cream or ointment bases and ethanol or oleyl alcohol for lotions. Such carriers may be present as from about 1% up to about 98% of the formulation. The ointment base can comprise one or more of petrolatum, mineral oil, ceresin, lanolin alcohol, panthenol, glycerin, bisabolol, cocoa butter and the like.

In some embodiments of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, preferably do not unduly interfere with the biological activities of the components of the compositions of the present invention. The formulations can be sterilized and, if desired, mixed with auxiliary agents (e.g., lubricants, preservatives, stabilizers, wetting agents, emulsifiers, salts for influencing osmotic pressure, buffers, colorings, flavorings and/or aromatic substances and the like) that do not deleteriously interact with the lantibiotic-based spore decontaminant of the formulation.

In some embodiments, the invention provides pharmaceutical compositions containing (a) a lantibiotic-based spore decontaminant; and (b) one or more other agents (e.g., an antibiotic). Examples of other types of antibiotics include, but are not limited to, almecillin, amdinocillin, amikacin, amoxicillin, amphomycin, amphotericin B, ampicillin, azacitidine, azaserine, azithromycin, azlocillin, aztreonam; bacampicillin, bacitracin, benzyl penicilloyl-polylysine, bleomycin, candicidin, capreomycin, carbenicillin, cefaclor, cefadroxil, cefamandole, cefazoline, cefdinir, cefepime, cefixime, cefinenoxime, cefinetazole, cefodizime, cefonicid, cefoperazone, ceforanide, cefotaxime, cefotetan, cefotiam, cefoxitin, cefpiramide, cefpodoxime, cefprozil, cefsulodin, ceftazidime, ceftibuten, ceftizoxime, ceftriaxone, cefuroxime, cephacetrile, cephalexin, cephaloglycin, cephaloridine, cephalothin, cephapirin, cephradine, chloramphenicol, chlortetracycline, cilastatin, cinnamycin, ciprofloxacin, clarithromycin, clavulanic acid, clindamycin, clioquinol, cloxacillin, colistimethate, colistin, cyclacillin, cycloserine, cyclosporine, cyclo-(Leu-Pro), dactinomycin, dalbavancin, dalfopristin, daptomycin, daunorubicin, demeclocycline, detorubicin, dicloxacillin, dihydrostreptomycin, dirithromycin, doxorubicin, doxycycline, epirubicin, erythromycin, eveminomycin, floxacillin, fosfomycin, fusidic acid, gemifloxacin, gentamycin, gramicidin, griseofulvin, hetacillin, idarubicin, imipenem, iseganan, ivermectin, kanamycin, laspartomycin, linezolid, linocomycin, loracarbef, magainin, meclocycline, meropenem, methacycline, methicillin, mezlocillin, minocycline, mitomycin, moenomycin, moxalactam, moxifloxacin, mycophenolic acid, nafcillin, natamycin, neomycin, netilmicin, niphimycin, nitrofurantoin, novobiocin, oleandomycin, oritavancin, oxacillin, oxytetracycline, paromomycin, penicillamine, penicillin G, penicillin V, phenethicillin, piperacillin, plicamycin, polymyxin B, pristinamycin, quinupristin, rifabutin, rifampin, rifamycin, rolitetracycline, sisomicin, spectrinomycin, streptomycin, streptozocin, sulbactam, sultamicillin, tacrolimus, tazobactam, teicoplanin, telithromycin, tetracycline, ticarcillin, tigecycline, tobramycin, troleandomycin, tunicamycin, tyrthricin, vancomycin, vidarabine, viomycin, virginiamycin, BMS-284,756, L-749,345, ER-35,786, S-4661, L-786,392, MC-02479, Pep5, RP 59500, and TD-6424. In some embodiments, two or more combined agents (e.g., a composition comprising a lantibiotic-based spore decontaminant and another antibiotic) may be used together or sequentially. In some embodiments, another antibiotic may comprise bacteriocins, type A lantibiotics, type B lantibiotics, liposidomycins, mureidomycins, alanoylcholines, quinolines, eveminomycins, glycylcyclines, carbapenems, cephalosporins, streptogramins, oxazolidonones, tetracyclines, cyclothialidines, bioxalomycins, cationic peptides, and/or protegrins. In some embodiments, a lantibiotic-based spore decontaminant comprises lysostaphin. In some embodiments, a lantibiotic-based spore decontaminant comprises mupirocin. In some embodiments, a lantibiotic-based spore decontaminant comprises one or more anti-anthrax agents (e.g., an antibiotic used in the art for treating B. anthracis (e.g., penicillin, ciprofloxacin, doxycycline, erythromycin, and vancomycin)).

The present invention also includes methods involving co-administration of a lantibiotic-based spore decontaminant with one or more additional active agents (e.g., an antibiotic, anti-oxidant, etc.). Indeed, it is a further aspect of this invention to provide methods for enhancing prior art therapies and/or pharmaceutical compositions by co-administering a composition comprising a lantibiotic-based spore decontaminant. In co-administration procedures, the agents may be administered concurrently or sequentially. In one embodiment, the compounds described herein are administered prior to the other active agent(s). The pharmaceutical formulations and modes of administration may be any of those described herein. In addition, the two or more co-administered agents may each be administered using different modes or different formulations. The additional agents to be co-administered, such as other antibiotics, can be any of the well-known agents in the art, including, but not limited to, those that are currently in clinical use.

In some embodiments, a lantibiotic-based spore decontaminant is administered to a subject via more than one route. For example, a subject that has been exposed to bacterial spores (e.g., B. anthracis spores) may benefit from receiving topical administration (e.g., via a spray, wipe, shower, bath, or other routes described herein) and, additionally, receiving pulmonary administration (e.g., via a nebulizer, inhaler, or other methods described herein). In some embodiments, a subject exposed to bacterial spores (e.g., B. anthracis spores) will have spores present on the skin, as well as within the airways. Thus, although an understanding of the mechanism is not necessary to practice the present invention and the present invention is not limited to any particular mechanism of action, it is contemplated that such a subject will benefit from multiple forms of treatment (e.g., topical as well as airway administration of a lantibiotic-based spore decontaminate of the present invention).

In some embodiments, pharmaceutical preparations comprising a lantibiotic-based spore decontaminant are formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of the compositions comprising a lantibiotic-based spore decontaminant (e.g., nisin) calculated to produce the desired antibacterial or sporicidal (e.g., killing or growth attenuation of bacterial spores) effect in association with the selected pharmaceutical carrier. Procedures for determining the appropriate dosage unit are well known to those skilled in the art.

Dosage units may be proportionately increased or decreased based on several factors (e.g., the duration of exposure or the magnitude of bacterial spore exposure (e.g., B. anthracis spore exposure), or, the weight of the subject. Appropriate concentrations for achieving eradication of pathogenic bacterial spores on a surface (e.g., skin or hair) may be determined by dosage concentration curve calculations, as known in the art.

In some embodiments, the composition comprises from 0.1 to 2000 μg/mL of lantibiotic (e.g., nisin). In some embodiments, the composition comprises from 2000 to 5000 μg/mL of lantibiotic (e.g., nisin). In some embodiments, a lantibiotic-based spore decontaminate of the present invention comprises 600 μg/mL of nisin. In some embodiments, the composition is from 0.01 to 15% or more (e.g., 0.1-10%, 0.5-5%, 1-3%, 2%, 6%, 10%, 15% or more) by weight lantibiotic (e.g., nisin). In some embodiments, the amount of lantibiotic (e.g., nisin) delivered to a subject is from 0.1 to 1000 mg/kg/day (e.g., 1 to 500 mg/kg/day, 5 to 250 mg/kg/day, 10-100 mg/kg/day, etc.).

It is contemplated that the compositions and methods of the present invention will find use in various settings, including research settings. For example, compositions and methods of the present invention also find use in studies of antibiotic resistance (e.g., via analysis of proteins and pharmaceuticals capable of altering antibiotic resistance) and in in vivo studies to observe susceptibility of bacterial cells or spores to antibacterial treatments. Uses of the compositions and methods provided by the present invention encompass human and non-human subjects and samples from those subjects, and also encompass research applications using these subjects. Thus, it is not intended that the present invention be limited to any particular subject and/or application setting.

A lantibiotic-based spore decontaminant of the present invention find use where the nature of the infectious spores present or to be avoided is known, as well as where the nature of the infectious spores is unknown. For example, the present invention contemplates use of the compositions of the present invention in treatment of or prevention of infections associated with any sporulating bacteria.

In some embodiments, pharmaceutical compositions of the present invention may be formulated for administration by oral (solid or liquid), parenteral (intramuscular, intraperitoneal, intravenous (IV) or subcutaneous injection), transdermal (either passively or using iontophoresis or electroporation), transmucosal (nasal, vaginal, rectal, or sublingual), or inhalation routes of administration, or using bioerodible inserts and can be formulated in dosage forms appropriate for each route of administration.

In a further embodiment of the invention, pharmaceutical compositions of the present invention can be used to prevent the infectious spread of bacterial spores (e.g., Clostridum difficile spores) in fecal material or fecally contaminated surfaces (e.g., human skin). In some embodiments, the present invention provides an antiseptic wipe designed to neutralize infectious spores shed in feces.

In a preferred embodiment, the compositions are administered by pulmonary delivery. For example, a composition of the present invention can be delivered to the lungs of a mammal (e.g., a human) via inhalation (e.g., thereby traversing across the lung epithelial lining to the blood stream (See, e.g., Adjei, et al. Pharmaceutical Research 1990; 7:565-569; Adjei, et al. Int. J. Pharmaceutics 1990; 63:135-144; Braquet, et al. J. Cardiovascular Pharmacology 1989 143-146; Hubbard, et al. (1989) Annals of Internal Medicine, Vol. III pp. 206-212; Smith, et al. J. Clin. Invest. 1989;84:1145-1146; Oswein, et al. “Aerosolization of Proteins”, 1990; Proceedings of Symposium on Respiratory Drug Delivery II Keystone, Col.; Debs, et al. J. Immunol. 1988; 140:3482-3488; and U.S. Pat. No. 5,284,656 to Platz, et al. A method and composition for pulmonary delivery of drugs for systemic effect is described in U.S. Pat. No. 5,451,569 to Wong, et al., hereby incorporated by reference; See also U.S. Pat. No. 6,651,655 to Licalsi et al., hereby incorporated by reference in its entirety)). The composition of the present invention may also be delivered with the intention of neutralizing spores or killing vegetative cells in the lungs either prior to uptake by phagocytic cells or within local phagocytic cells.

Further contemplated for use in the practice of this invention are a wide range of mechanical devices designed for pulmonary delivery of therapeutic agents (e.g., a lantibiotic-based spore decontaminant), including but not limited to nebulizers, metered dose inhalers, and powder inhalers, all of which are familiar to those skilled in the art. Some specific examples of commercially available devices suitable for the practice of this invention are the Ultravent nebulizer (Mallinckrodt Inc., St. Louis, Mo.); the Acorn II nebulizer (Marquest Medical Products, Englewood, Colo.); the Ventolin metered dose inhaler (Glaxo Inc., Research Triangle Park, N.C.); and the Spinhaler powder inhaler (Fisons Corp., Bedford, Mass.). All such devices require the use of formulations suitable for the dispensing of the therapeutic agent. Typically, each formulation is specific to the type of device employed and may involve the use of an appropriate propellant material, in addition to the usual diluents, adjuvants, surfactants and/or carriers useful in therapy. Also, the use of liposomes, microcapsules or microspheres, inclusion complexes, or other types of carriers is contemplated.

Formulations for use with a metered-dose inhaler device will generally comprise a finely divided powder containing the therapeutic agent suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

In some embodiments, formulations for dispensing from a powder inhaler device will comprise a finely divided dry powder containing the therapeutic agent, and may also include a bulking agent, such as lactose, sorbitol, sucrose, or mannitol in amounts which facilitate dispersal of the powder from the device (e.g., 50 to 90% by weight of the formulation). The therapeutic agent should most advantageously be prepared in particulate form with an average particle size of less than 10 mm (or microns), most preferably 0.5 to 5 mm, for most effective delivery to the distal lung.

Nasal or other mucosal delivery of the therapeutic agent is also contemplated. Nasal delivery allows the passage to the blood stream directly after administering the composition to the nose, without the necessity for deposition of the product in the lung. Formulations for nasal delivery include those with dextran or cyclodextran and saponin as an adjuvant. Nasal delivery has further benefit of neutralizing spores present in the nasal passage (See, e.g., Example 13). Delivery of the therapeutic agent to the nasal passages may also have the added benefit of neutralizing spores in the nasal passages before they can cause localized infection or passage to the lungs to cause wide spread infection.

A composition of the present invention may be administered in conjunction with one or more additional active ingredients, pharmaceutical compositions, or vaccines.

### EXPERIMENTAL

The following examples are provided in order to demonstrate and further illustrate certain preferred embodiments and aspects of the present invention and are not to be construed as limiting the scope thereof.

### EXAMPLE 1

### Nisin Activity Against Vegetative Bacilli and other Organisms

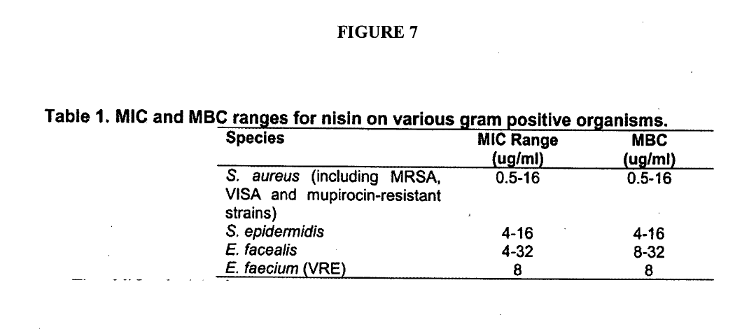

Nisin is active against a wide range of gram positive organism (See, e.g., Table 1 shown in FIG. 7). The minimum inhibitory concentration (MIC) and minimum bactericidal concentrations (MBC) for organisms listed in Table 1 were determined by standard methods (See, e.g., National Committee for Clinical Laboratory Standards, 1997, Villanova, Pa., 4th Edition) except that a higher initial inoculum was used to facilitate MBC determination.

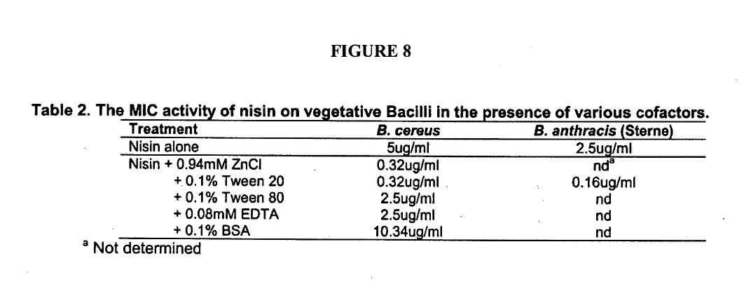

The MIC of nisin for vegetative B. cereus and B. anthracis (Sterne) cells was also determined by microbroth dilution assay using BHI media in the absence or presence of various potential cofactors (See, e.g., Table 2 shown in FIG. 8). These studies demonstrated a synergistic enhancement of nisin's antibacterial activity in the presence of various factors including chelators, surfactants, and essential oils.

### EXAMPLE 2

### Nisin's Activity against Bacterial Spores

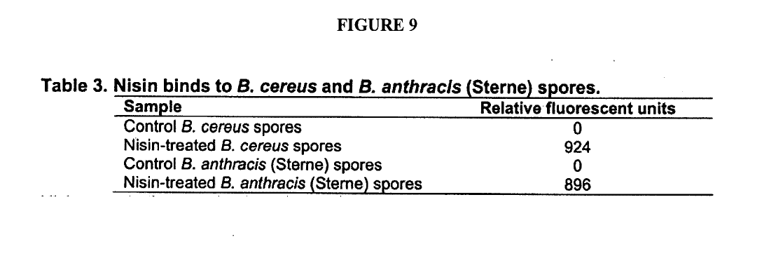

The capacity of nisin to bind to and prevent the outgrowth of bacilli spores was examined under various conditions. It was demonstrated that nisin binds to spores of B. cereus and B. anthracis. Spores of B. cereus and B. anthracis were prepared by the method of Tisa et al (See, e.g., Tisa et al., 1982 Appl. Environ. Microbiol. 6, 550-556) and purified on a Percoll gradient which resulted in spores free of mother cell contamination. These highly purified spores were incubated with 600 μg/ml nisin +0.1% Tween 20 for 10 minutes and then washed by spin filtration. Nisin-treated spores, or untreated control spores, were then incubated with Alexaflour labeled, affinity purified sheep anti-nisin (Ambi, Purchase, N.Y.). Spores were then washed and fluorescence determined. Nisin binds to spores of both B. cereus and B. anthracis and remains bound despite extensive washing (See, e.g., Table 3 shown in FIG. 9).

### EXAMPLE 3

### Nisin Arrests the Germination of B. cereus and B. anthracis Spores In Vitro

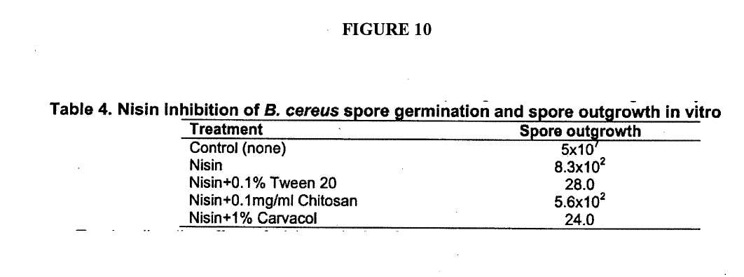

Highly purifies pores of B. cereus were treated with various concentrations of nisin for 10 minutes and then washed in PBS. The washed spores were serially diluted and incubated on brain heart infusion agar (BHI) plates overnight. Table 4 shown in FIG. 10 shows a 5 log₁₀ reduction in spore germination and outgrowth following nisin treatment of spores. The addition of surfactants (e.g., Tween-20 and Carvacol (2-p-cymenol, an essential oil extracted from oregano and thyme)) further reduced spore germination and outgrowth by 2 log₁₀.

In order to visualize the effect of nisin on isolated spores, highly purified spores of B. anthracis (Sterne) were treated with 600 μg/ml nisin for 10 minutes, washed, and then incubated in BHI +5% glycerol broth. Samples were taken from the BHI cultures at various time points and spores and/or vegetative cells were examined under phase microscopy. Visual examination of the Sterne spores revealed that all spores, whether treated or not with nisin, progressed from phase bright (at 5 minutes) to phase dark (at 1 hour). Most notable, however, was the observation that vegetative growth could be observed by 2 hours in the untreated spores, but nisin treated spores remained phase dark spores for the entire 24 hour period of observation.

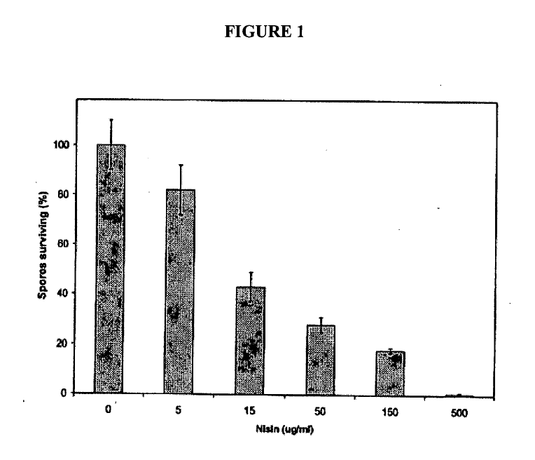

The Sterne strain of B. anthracis does not carry the pXO2 plasmid that comprises genes that encode for production of capsule (See, e.g., Mock and Fouet, 2001, Anthrax Annu Rev Microbiol 55, 647-671). In order to determine whether nisin had the same effect on spores from a fully virulent strain of B. anthracis, B. anthracis (Ames) spores (350/ml) were exposed to various concentrations of nisin in water for 10 minutes on ice. The spores were then pelleted by centrifugation, resuspended in water, serially diluted and then plated on tryptic soy agar. As shown in FIG. 1, there was a dose responsive inhibition of the outgrowth of the B. anthracis (Ames) spores with increasing concentrations of nisin.

### EXAMPLE 4

### Nisin Neutralized Spores Remain Attenuated Over Time

It is important that nisin-treated spores remain inert for long periods of time after treatment. In order to determine whether this occurred in vitro, sample of spores (˜5×10⁷/ml) were treated in Na citrate buffer at room temperature for 10 min with nisin as described above. Two of the samples were washed by spin filtration and resuspended in sterile buffer and two remained in the nisin in buffer to serve as a positive control for neutralization of spores. One washed and one unwashed nisin-treated spore sample along with a buffer-treated control sample of spores were placed at room temperature or in a 37° C. incubator. At various time points following nisin-treatment, including days 0, 3, 7, 14, 28 and 56, an aliquot of each sample was taken from the tubes, washed by spin filtration, and the germination capacity of each of the spore samples determined as described above.

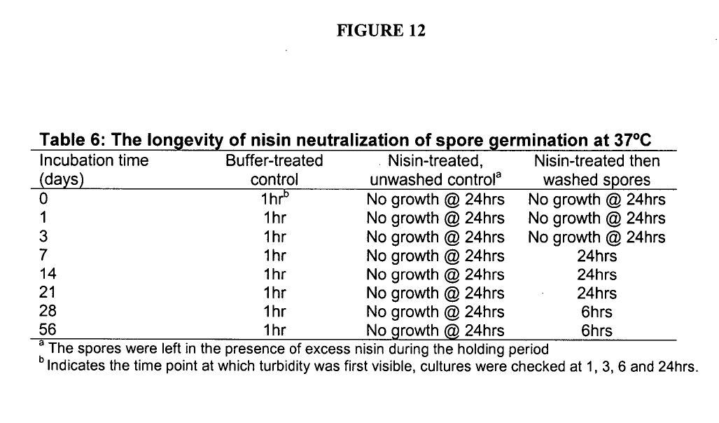

As shown in Table 6, FIG. 12, nisin treated spores remained completely neutralized at 37° C. through day 3 (as determined by lack of growth at 24 hrs post inoculation of BHI media). Starting on day 7, some turbid growth was noted after 24 hrs of incubation of the BHI media. This delay in visible turbid growth was further reduced to 6 hrs following 28 days of incubation of the washed, nisin-treated spores at 37° C. However, nisin-treated spores incubated for 56 days at 37° C. continued to demonstrate delayed growth (6 versus 1 hr to visible growth) indicating that nisin continues to affect spores even for extended time periods. Buffer-treated control spores had visible growth at 1 hr post inoculation of BHI media throughout the experiment, while the positive control sample where spores were left in the 500 μg/ml nisin solution did not germinate and grow even after 56 days of incubation at 37° C. This indicates that higher concentrations of nisin remain stable throughout this experiment. When this experiment was conducted at room temperature, similar results were seen.

### EXAMPLE 5

### Nisin-Treated Spores do not Germinate in Macrophages

Following entry of B. anthracis spores into the body by inhalation, ingestion or wound contamination, spores are phagocytosed by local macrophages and germination of the spores begins (See, e.g., Dixon et al., 2000 Cell Microbiol 2, 453-463; Guidi-Rontani 2002 Trends Microbiol 10, 405-409). In order to determine whether nisin-treated spores are blocked for germination in macrophages, a cell culture infection system was developed. Briefly, B. anthracis (Sterne) spores were fluorescently tagged with Alexaflour to allow their visualization by fluorescent microscopy. Spores were treated in the presence or absence of 600 μg/ml nisin for 15 minutes and then washed. The human macrophage cell line RAW-264.7 was grown on chamber slides and then incubated with nisin-treated or untreated B. anthracis (Sterne) spores at an MOI of 4 for 2 hours. Following the incubation period, the cell monolayer was washed to remove free spores. Uptake of spores by the RAW-264.7 cells was visualized at various time points by a combination of light and fluorescent microscopy. The challenged RAW-264.7 cells were then observed at various time points after infection. Vegetative growth of the B. anthracis (Sterne) cells could be clearly seen as filamentous strands in RAW-264.7 cell cultures challenged with untreated spores within 2 hours, whereas the nisin-treated spores remained as inert spores up to 7 hours (when the experiment was terminated).

### EXAMPLE 6

### Pretreatment of Spores with Nisin Protects Mice from Death in a Mouse Intrapulmonary Challenge Model

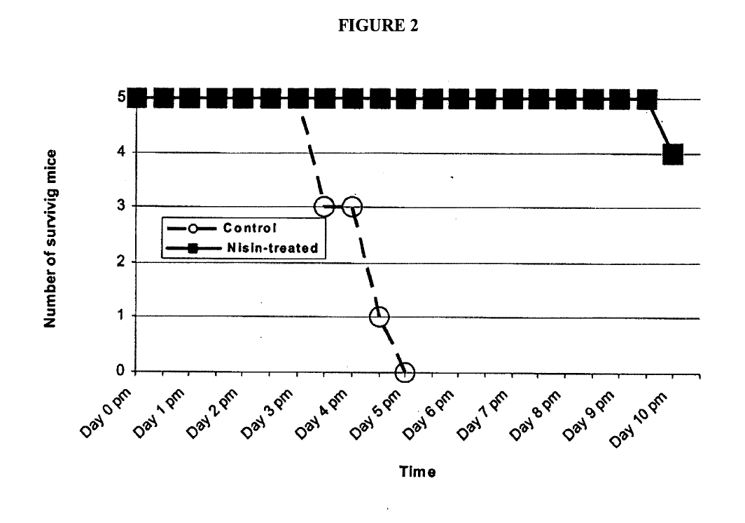

A/J mice are susceptible to B. anthracis (Sterne) spore challenge (See, e.g., Friedlander et al., 1993 Infect Immun 61, 245-252). Intranasal instillation of spores in buffer leads to spores reaching the alveoli where they are taken-up by alveolar macrophages, germinate, express toxins and eventually lead to death of the animal over several days (See, e.g., Mock and Fouet, Anthrax Annu Rev Microbiol 2001 55, 647-671). When A/J mice were challenged with 1.1×10⁵ B. anthracis (Sterne) spores, all five animals in the group succumbed to the infection by day 5 (See FIG. 2). However, when mice were challenged with spores that had been treated with nisin (600 μg/ml) for 15 minutes and then washed, only a single death was observed (on day 10) with no further deaths through day 20.

### EXAMPLE 7

### Nisin-Treated Spores Remain Neutralized for Long Periods In Vivo

During the anthrax attacks of 2001, people were given a 60 day course of antibiotics to ensure that they were protected against late emergence of vegetative B. anthracis from phagocytosed spores. Given that spores can remain inert within macrophages over extended periods, it was important to determine whether nisin-treated spores remain inert over an extended time and whether nisin leads to germination arrest and not simply to delayed onset of vegetative growth. It has previously been shown that treatment of Sterne spores and variant Cipro-R spores greatly attenuates these spores in an A/J mouse intrapulmonary challenge model. A/J mice are known to be susceptible to challenge by the capsuleless Sterne strain of B. anthracis, and A/J mice were used as a model for pulmonary challenge with nisin-treated spores.

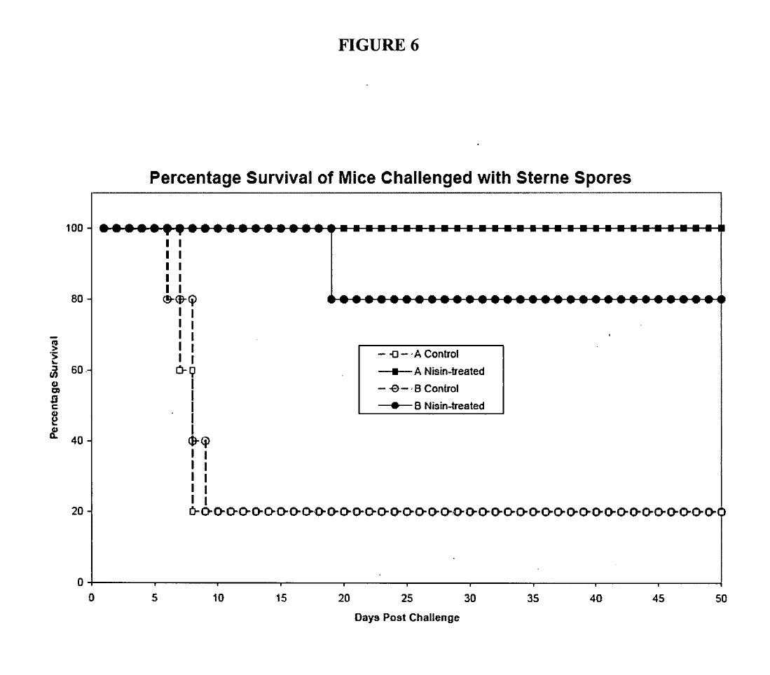

Separate populations of spores were generated by either growing B. anthracis (Sterne) on solid media and allowed to go to sporulation (referred to as type A spores in FIG. 6) or growing B. anthracis (Sterne, different original stock than that grown on solid media) in liquid media and allowed to go to sporulation (referred to as type B spores in FIG. 6). Both spore preps were purified using gradient centrifugation. Spores were treated with either 500 μg/ml nisin in buffer or buffer alone for 10 min. Following treatment, the spores were washed twice and then resuspended in water. A/J mice were anesthetized and then challenged intranasally with ˜5×10⁶ of treated or untreated spores in 50 μl volume. The animals were then monitored for lethality over several weeks. As shown in FIG. 6, all of the mice challenged with nisin-treated type A spores, and 80% of the mice challenged with nisin-treated type B spores survived for 55 days, while 80% of the mice challenged with either buffer-treated control spore preparation succumbed to their infection within 10 days. The one mouse that did succumb to nisin-treated spores did not do so until nearly day 20.

Thus, the present invention provides that spores produced two different ways from two different seed stocks are neutralized by nisin in vivo, that nisin treated spores are greatly attenuated/neutralized when inhaled, that nisin treated spores remain attenuated/neutralized over a prolonged time after administration to the lungs, and that nisin treatment of spores does not merely delay the onset of disease.

### EXAMPLE 8

### Selection of Ciprofloxacin Resistant B. anthracis Variants and Testing Nisin for Spore Neutralization and Attenuation of Virulence

During the anthrax-by-mail attacks of 2001, Ciprofloxacin (Cipro) was the antibiotic of choice for those who developed anthrax and for those potentially exposed to spores (See, e.g., Frist, 2002, When Every Moment Counts, What You Need to Know About Bioterrorism, Rowman and Littlefield Publishers, Inc. NY). Many people were put on sixty day courses of the antibiotic if exposure was even suspected. Resistance to Cipro can be selected in B. anthracis by culturing the bacteria in the presence of increasing concentrations of the antibiotic (See, e.g., Athamna et al., 2003 J Antimicrob Chemother 54, 424-428). During the development of the present invention, and using the aforementioned process, B. anthracis (Sterne) variants that are resistant to 8 mg/L Cipro were isolated.

Spores of this resistant strain were produced (Cipro-R). It was determined that spores of Cipro-R B. anthracis were also blocked by nisin from germinating in vitro in the same manner as Cipro-sensitive spores. Furthermore, while spores of Cipro-R bacteria were less pathogenic in the mouse pulmonary challenge model (lethal dose ˜10⁷ Cipro-R spores versus ˜10⁵ for parental strain spores), nisin also attenuated the Cipro-R spores in this model (e.g., 8 of 10 control mice succumbed to infection, while 2 of 10 mice challenged with nisin-treated spores died at greater than 20 days after exposure). The specter of an attack using Cipro-R spores is frightening since there would be a delay of several days before resistance to Cipro was determined. The present invention provides an alternative defense.

### EXAMPLE 9

### B. anthracis (Sterne) Spore Wound Infection Model

A hairless mouse skin contamination models was developed using SKH mice (See, e.g., ASM General Meeting Abstract. S. Walsh, A. Shah, J. Mond, Abstract # A-021. Meeting dates 18-22 May, 2003, Washington D.C.

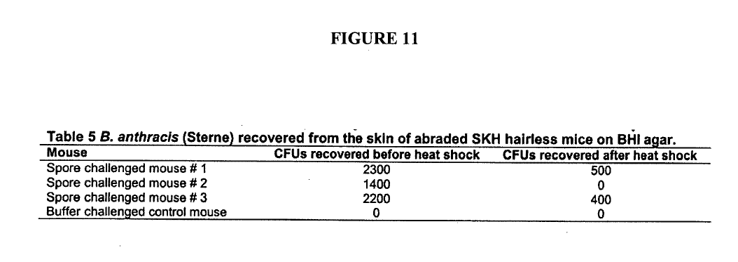

Using B. anthracis (Sterne) spores, the skin on the backs of 4 SKH hairless mice was abraded with sterilized 150 grit sand paper and ˜100 μl of a solution containing 10⁷ spores/ml was swabbed on the skin. Four days after challenge, the affected skin was sampled by swabbing with a swab wet in PBS. The bacteria on the swabs were resuspended in PBS and plated on BHI agar and blood agar to enumerate recovered bacteria. These same buffer solutions were then heat shocked at 70° C. for 15 minutes and plated again. Table 5 in FIG. 11 shows the results of the recovery.

The recovered counts were almost identical on blood agar and only B. anthracis (Sterne) was recovered on either agar. These results indicate that most of the applied spores had germinated and were growing as vegetative cells in the wounds, but in two of the mice, spores were also recovered from the wounds.

### EXAMPLE 10

### Nisin-Neutralization of B. anthracis (Sterne) Spores on a Surface

80 μl (˜10⁷) of either B. anthracis (Sterne) or Cipro-R Sterne spores were pipetted onto a glass slide. The spore suspension was allowed to air dry to form an adherent spore spot. 150 μl of either a 0.6 mg/ml nisin solution or buffer control was pipetted onto the died spore spots and incubated for 15 minutes at room temperature. The solution was removed and the spots washed 3× with water. Following removal of the final water wash, 150 μl of BHI media was added to each spore spot and the resuspended spores were placed in a culture tube containing BHI media using a sterile cell scraper. Spores were incubated with shaking at 37° C. Aliquots were removed at various time points for microscopic observation to determine the germination and vegetative state of the spores.

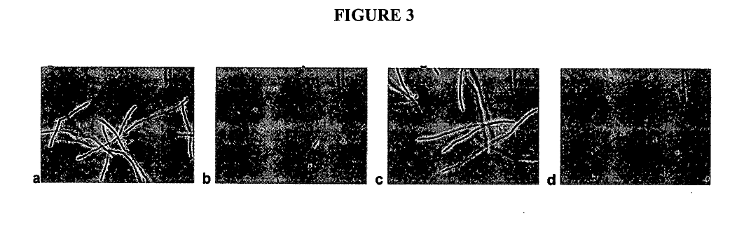

FIG. 3 shows an aliquot from the final time point (5 hours). Vegetative growth of the buffer treated Sterne (a) and Cipro-R Sterne (c) spores was clearly visible (e.g., growth first seen at 20 minutes), nisin pretreated spores of both types (b and d, respectively) remain inert throughout the experiment.

### EXAMPLE 11

### Spore Neutralization on a Surface by Nisin-Impregnated Wipe

Squares of untreated sterile disposable wipe material (25 mm×25 mm) were impregnated with either a 0.6 mg/ml nisin solution or buffer alone and then the excess buffer or nisin solution was removed to create damp wipes. Dried spore spots of B. anthracis (Sterne) spores were deposited on glass slides as described in Example 10 above. The dried spore spots were then wiped vigorously with either the nisin-impregnated or buffer-impregnated wipe (microscopy revealed that this treatment removed most of the spores from the glass slide). The whole wipe was then transferred to a tube containing sterile water, briefly sonicated to release adhering spores, and an aliquot of the water was used to inoculate BHI media which was incubated overnight with shaking at 37° C. Following 18 hours of incubation, the BHI cultures were observed for vegetative growth based on turbidity.

The spores wiped with buffer-impregnated wipes resulted in turbid growth of vegetative B. anthracis (confirmed by gram stain and culture) while the culture inoculated with spores wiped with nisin-impregnated wipes remained clear at 18 hours post inoculation. Thus, the present invention demonstrates that nisin-impregnated wipes can effectively neutralize B. anthracis spores on a surface. The process of sonicating the wipes in water and then using the water to inoculate BHI media minimized the residual carry-over of nisin such that the concentration of nisin present in the media is too low to effectively neutralize spores following inoculation. Thus, although an understanding of the mechanism is not necessary to practice the present invention and the present invention is not limited to any particular mechanism of action, in some embodiments, nisin-impregnated wipes are capable of neutralizing spores upon contact.

### EXAMPLE 12

### Nisin Neutralizes B. anthracis Spores that Have Already been Phagocytosed by Macrophages

To determine if post spore-uptake use of nisin would be beneficial in addition to nisin's neutralization of spores on skin, it was determined whether nisin could penetrate macrophages to neutralize spores after phagocytosis or post-inhalation. Macrophages (RAW-264.7 cells) were pre-treated with B. anthracis (Sterne) spores at an MOI of 10 spores/cell for 1.5 hrs. The cell monolayers were than washed twice to remove free spores, and the macrophages treated with either media alone or media containing 0.5 mg/ml nisin for 1 hr. Following this incubation, the cells were washed twice to remove excess nisin and fresh media was added. The macrophage cell culture was microscopically observed at various time points for uptake of spores and subsequent vegetative growth of B. anthracis.

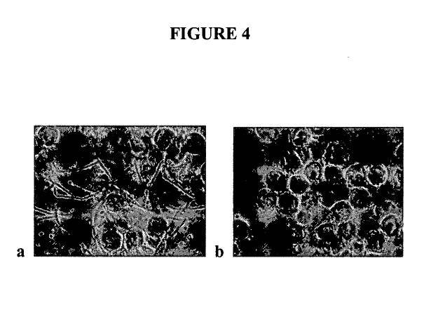

Vegetative growth of B. anthracis from spores phagocytosed by control cells was observed whereas no growth was observed in nisin-treated cells five hours after uptake (See FIG. 4). Thus, the present invention demonstrates that a lantibiotic (e.g., nisin) can be used to neutralize spores already phagocytosed by macrophages (e.g., the lantibiotic can penetrate the macrophages).

### EXAMPLE 13

### Post-Inhalational Spore Neutralization by Intranasal Application of Nisin

A/J mice were nasally instilled with either 5×10⁵ B. anthracis (Sterne) spores (two groups) or 5×10⁵ spores pre-treated with nisin (0.6 mg/ml). Four hours post instillation, one group of spore-challenged mice received a 50 μl nasal instillation of 0.6 mg/ml nisin in buffer. The mice were then followed for lethality.

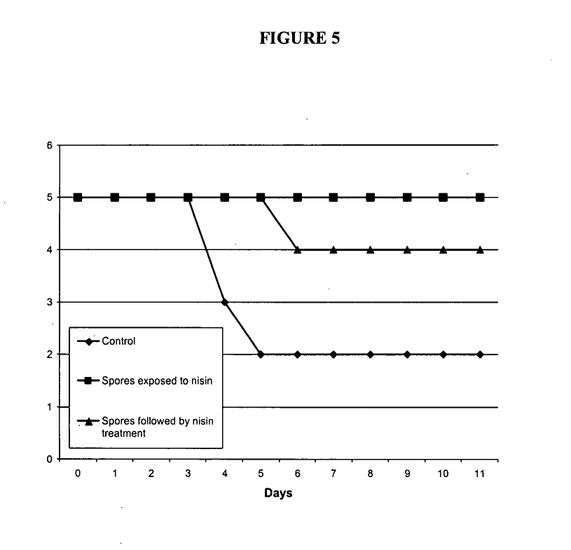

Monitoring of the mice for 11 days demonstrated that 3 of 5 spore challenged mice succumbed to their infection by day 4 post challenge while all five of the mice challenged with nisin-treated spores survived through day 11. Nisin treatment four hours post spore challenge, protected 4 of 5 mice from lethality to day 11 (See FIG. 5). Thus, the present invention demonstrates that a lantibiotic (e.g., nisin) can be used to neutralize spores in vivo (e.g., nisin can penetrate eukaryotic cells (e.g., nisin can be used as a post exposure treatment for inhaled anthrax spores (e.g., to neutralize spores not neutralized on the skin))).

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method of treating a subject exposed to bacterial spores comprising administering to said subject a lantibiotic-based spore decontaminant under conditions such that said spores are neutralized.

2. The method of claim 1, wherein said spores are Bacillus anthracis spores.

3. The method of claim 2, wherein said Bacillus anthracis spores are derived from antibiotic resistant bacteria.

4. The method of claim 3, wherein said antibiotic resistant bacteria are ciprofloxacin resistant.

5. The method of claim 1, wherein said spores are Clostridium difficile spores.

6. The method of claim 2, wherein said Bacillus anthracis spores are released during a terrorist attack.

7. The method of claim 2, wherein said Bacillus anthracis spores are released in a laboratory.

8. The method of claim 5, wherein said spores are contaminants associated with an outbreak of Clostridium difficile colitis.

9. The method of claim 5, wherein said lantibiotic-based spore decontaminant is administered in a setting selected from the group consisting of a daycare, a hospital, a nursing home, and a school.

10. The method of claim 1, wherein said lantibiotic-based decontaminant comprises nisin.

11. The method of claim 1, wherein said lantibiotic-based spore decontaminant is administered to said subject via a spray.

12. The method of claim 1, wherein said lantibiotic-based spore decontaminant is administered to said subject via a foam.

13. The method of claim 1, wherein said lantibiotic-based spore decontaminant is administered to said subject via a cream.

14. The method of claim 1, wherein said lantibiotic-based spore decontaminant is administered to said subject via a disposable wipe impregnated with said lantibiotic.

15. The method of claim 14, wherein said disposable wipe is used to decontaminate skin exposed to fecal material containing Clostridium difficile spores.

16. The method of claim 1, wherein said lantibiotic-based spore decontaminant is administered to said subject via a shower.

17. The method of claim 1, wherein said subject is administered said lantibiotic-based spore decontaminant by submersion of said subject in a solution of said decontaminant.

18. The method of claim 1, wherein said lantibiotic-based spore decontaminant is administered to said subject via pulmonary administration.

19. The method of claim 18, wherein said pulmonary administration comprises inhalation of said lantibiotic-based spore decontaminant by said subject.

20. The method of claim 18, wherein said pulmonary administration comprises use of a nebulizer.

21. The method of claim 18, wherein said pulmonary administration comprises use of a metered dose inhaler.

22. The method of claim 18, wherein said pulmonary administration comprises use of a powder inhaler.

23. The method of claim 18, wherein said pulmonary administration is administered to said subject within 48 hours after exposure to said spores.

24. The method of claim 18, wherein said pulmonary administration is administered to said subject within 12 hours after exposure to said spores.

25. The method of claim 18, wherein said pulmonary administration is administered to said subject within 4 hours after exposure to said spores.

26. The method of claim 1, wherein said lantibiotic-based spore decontaminant is administered to said subject via nasal administration.

27. The method of claim 1, wherein said lantibiotic-based spore decontaminant is administered to a mucosal surface of said subject.

28. The method of claim 1, wherein said lantibiotic-based spore decontaminant is administered in vivo.

29. The method of claim 1, wherein said subject is a human subject.

30. The method of claim 1, wherein said subject is one of a population of subjects exposed to said Bacillus anthracis spores.

31. The method of 30, wherein said population comprises between 10 and 10,000 human subjects.

32. The method of claim 1, wherein said lantibiotic-based spore decontaminant is administered to said subject via wiping said subject with a wipe moistened with said decontaminant.

33. The method of claim 1, wherein neutralizing said spores prohibits outgrowth of said spores.

34. The method of claim 1, wherein neutralizing said spores kills said spores.

35. The method of claim 1, wherein neutralizing said spores protects said subject from experiencing signs or symptoms of Bacillus anthracis infection.

36. The method of claim 1, wherein said lantibiotic-based spore decontaminant comprises a detergent polysorbate.

37. The method of claim 36, wherein said detergent polysorbate is Tween-20.

38. The method of claim 1, wherein said decontaminant is administered to said subject within 48 hours after exposure to said spores.

39. The method of claim 1, wherein said decontaminant is administered to said subject within 12 hours after exposure to said spores.

40. The method of claim 1, wherein said decontaminant is administered to said subject within 4 hours after exposure to said spores.

41. The method of claim 1, wherein said decontaminant is administered to said subject within 2 hours after exposure to said spores.

42. A method of preventing signs and symptoms of Bacillus anthracis infection in a subject comprising administering to said subject a lantibiotic-based spore decontaminant configured to neutralize Bacillus anthracis spores.

43. The method of claim 42, wherein said lantibiotic-based decontaminant comprises nisin.

44. The method of claim 42, wherein said lantibiotic-based spore decontaminant is administered to said subject via a spray.

45. The method of claim 42, wherein said lantibiotic-based spore decontaminant is administered to said subject via a foam.

46. The method of claim 42, wherein said lantibiotic-based spore decontaminant is administered to said subject via a cream.

47. The method of claim 42, wherein said lantibiotic-based spore decontaminant is administered to said subject via a disposable wipe.

48. The method of claim 42, wherein said lantibiotic-based spore decontaminant is administered to said subject via a shower.

49. The method of claim 42, wherein said subject is administered said lantibiotic-based spore decontaminant by submersion of said subject in a solution of said decontaminant.

50. The method of claim 42, wherein said lantibiotic-based spore decontaminant is administered to said subject via pulmonary administration.

51. The method of claim 50, wherein said pulmonary administration comprises inhalation of said lantibiotic-based spore decontaminant by said subject.

52. The method of claim 50, wherein said pulmonary administration comprises use of a nebulizer.

53. The method of claim 50, wherein said pulmonary administration comprises use of a metered dose inhaler.

54. The method of claim 50, wherein said pulmonary administration comprises use of a powder inhaler.

55. The method of claim 50, wherein said pulmonary administration is administered to said subject within 48 hours after exposure to said spores.

56. The method of claim 50, wherein said pulmonary administration is administered to said subject within 12 hours after exposure to said spores.

57. The method of claim 50, wherein said pulmonary administration is administered to said subject within 4 hours after exposure to said spores.

58. The method of claim 50, wherein said pulmonary administration is administered to said subject within 2 hours after exposure to said spores.

59. The method of claim 42, wherein said decontaminant is administered to said subject within 48 hours after exposure to said spores.

60. The method of claim 42, wherein said decontaminant is administered to said subject within 12 hours after exposure to said spores.

61. The method of claim 42, wherein said decontaminant is administered to said subject within 4 hours after exposure to said spores.

62. The method of claim 42, wherein said decontaminant is administered to said subject within 2 hours after exposure to said spores.

63. The method of claim 50, wherein said lantibiotic-based spore decontaminant is administered to said subject via nasal administration.

64. The method of claim 50, wherein said lantibiotic-based spore decontaminant is administered to a mucosal surface of said subject.

65. The method of claim 50, wherein said lantibiotic-based spore decontaminant is administered in vivo.

66. The method of claim 50, wherein said subject is a human subject.

67. A device configured for pulmonary administration comprising nisin.

68. The device of claim 67, wherein said device is a nebulizer.

69. The device of claim 67, wherein said device is a metered dose inhaler.

70. The device of claim 67, wherein said device is an inhaler.

71. The device of claim 70, wherein said inhaler is a powder inhaler.

72. A composition comprising nisin and an antibiotic used to treat anthrax, wherein said composition is configured for internal administration to a subject.

73. The composition of claim 72, wherein said antibiotic is ciprofloxacin.

74. The composition of claim 72, wherein said antibiotic is selected from the group consisting of penicillin, doxycycline, erythromycin, and vancomycin.
